(19) United States
(12) Patent Application Publication
(10) Pub. No.: US 2017/0213012 A1
(43) Pub. Date: Jul. 27, 2017

(54) SYSTEMS AND METHODS FOR CAPACITIVE IDENTIFICATION

(71) Applicant: CareFusion 303, Inc., San Diego, CA (US)

(72) Inventors: Cathal O'SCOLAI, Encinitas, CA (US); Mark Patrick BLOOM, Chula Vista, CA (US); David Patrick BROWN, San Diego, CA (US); Harry WEBER, Chula Vista, CA (US); Michael PESSOTTO, Escondido, CA (US)

(21) Appl. No.: 15/005,955

(22) Filed: Jan. 25, 2016

(51) Int. Cl. G06F 19/00; G06K 7/10; A61M 5/142

(52) U.S. Cl. CPC G06F 19/3468; A61M 5/142; G06K 7/10366; A61M 2205/3337; A61M 2205/3331; A61M 2205/6072

(57) ABSTRACT

Capacitive identification systems and methods are described. The system may include a capacitive detector configured to identify objects having capacitive identifiers. A capacitive identifier may be a patterned dielectric ink that, when placed in an electromagnetic field generated by the capacitive detector, detectably alters the capacitance of neighboring sensors in the detector. The capacitive detector may be disposed within an opaque housing structure of a device and configured to detect the capacitive identifier through the opaque housing structure. The capacitive detector may include a capacitive sensing array and processing circuitry formed on a common printed circuit. The capacitive detector may be formed in an infusion pump system. The capacitive identifier may be disposed on a pump cassette for the infusion pump system.

### TECHNICAL FIELD

The present disclosure generally relates to apparatus, systems, and methods of identification, and more particularly to capacitive identification systems and associated methods.

### BACKGROUND

Technological systems for electronic identification of objects are widespread and include optical scanners of barcodes or quick-response codes at retail stores and other locations and radio-frequency identification (RFID) tags that emit radio-frequency signals containing identifying information for objects such as livestock, consumer products, and shipping containers.

In some situations, accurate identification of an object can be critical. For example, infusion pumps are medical devices that may be used to administer intravenous (IV) fluids. An infusion pump can facilitate the delivery of IV fluids while controlling the volumes and rates for the delivery of such IV fluids. A typical infusion pump manipulates an IV tube or IV cartridge such that the IV fluid moves from a container to a patient. The IV tube or IV cartridge is typically connected to or integrated with an IV set (e.g., tubing, valves, filter, check valves, injection ports, and fittings for delivering fluid to a patient), and therefore the cartridge and IV set may be disposable to reduce the risk of infection and contamination. Thus, identification of a particular disposable cartridge and IV set coupled to the pump may be important so that the IV fluids are properly delivered to the patient and medical errors are avoided.

Particularly for disposable objects, it would be desirable to be able to provide identification systems and methods that reduce the cost and complexity and/or improve the accuracy and reliability of object identification relative to conventional barcode and RFID systems.

### SUMMARY

Aspects of the subject technology relate to capacitive identification of objects. Some aspects of the subject technology relate to identification of disposable IV pump cassettes using infusion pump systems having capacitive detectors.

In accordance with certain aspects, an apparatus is provided that includes a capacitive sensing array; and processing circuitry coupled to the capacitive sensing array and configured to: operate the capacitive sensing array to generate an electromagnetic field; and determine an identity of an object based on capacitance values generated by the capacitive sensing array when a capacitive identifier of the object is placed within the electromagnetic field generated by the array.

In accordance with certain aspects, a pump cassette is provided that includes a rigid body comprising a compliant membrane that defines a controllable fluid pathway that extends from an inlet port to an outlet port; and a capacitive identifier comprising a coded pattern that identifies the pump cassette.

In accordance with certain aspects, an infusion pump system is provided that includes: a processing unit; and a cassette recess adapted to receive a pump cassette, the cassette recess comprising: a plurality of mechanisms operably coupled to the processing unit and configured to control fluid flow in the pump cassette; and a capacitive detector configured to capacitively detect and identify the pump cassette.

It is understood that in accordance with certain aspects, the cassette recess may be integrated into the same box as the processing unit or may be contained in an interface module that may be operatively coupled to the processing unit.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIGS. 1A and 1B are overview diagrams illustrating examples of infusion pump systems, in accordance with aspects of the present disclosure.

FIGS. 2A and 2B illustrate perspective views of examples of an embodiment of a disposable IV pump cassette and cassette recess, in accordance with aspects of the present disclosure.

FIG. 3 illustrates a perspective view of the example embodiment of the disposable IV pump cassette of FIGS. 2A and 2B, in accordance with aspects of the present disclosure.

FIG. 4 illustrates an example embodiment of a capacitive detector board, in accordance with aspects of the present disclosure.

FIG. 5 illustrates a block diagram of an example embodiment of a capacitive identification system having a capacitive detector and an object having a capacitive barcode, in accordance with aspects of the present disclosure.

FIG. 6 illustrates a cross-sectional view of an example embodiment of a portion of a cassette recess showing how a capacitive detector can be formed behind a housing structure of the cassette recess, in accordance with aspects of the present disclosure.

FIG. 7 illustrates a flowchart showing illustrative operations that may be performed for capacitive identification of objects, in accordance with aspects of the present disclosure.

FIG. 8 illustrates a flowchart showing illustrative operations that may be performed for gathering capacitance data, in accordance with aspects of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Various aspects of the present disclosure relate to capacitive identification systems. A capacitive identification system may include capacitive detector (e.g., a detector having one or more capacitive sensing arrays and associated processing circuitry for receiving and processing signals generated with the arrays) and one or more objects having capacitive identifiers (e.g., one or more patterned capacitive structures such as a barcode printed using dielectric ink in which the pattern includes coded identification information for the object). According to various embodiments, capacitive identification systems may be provided in medical systems such as infusion pump systems or drug tracking systems, retail product tracking systems, shipping container tracking systems, or other systems in which objects having capacitive identifiers can be detected, identified, and tracked.

Capacitive sensors may be positioned within a device and behind an enclosure wall of the device, particularly because (in contrast with an optical scanner for a conventional optical barcode), a capacitive sensor may be operable to detect and identify a capacitive barcode through the enclosure wall without a direct optical view of the barcode. In this way, a barcode reader may be provided with improved resistance to ingress of fluids, dust, or other contaminants. This can be particularly beneficial in applications in which a barcode needs to be read but the reader needs to be sealed against fluid ingress, such as a wide range of medical devices and other industrial/commercial systems.

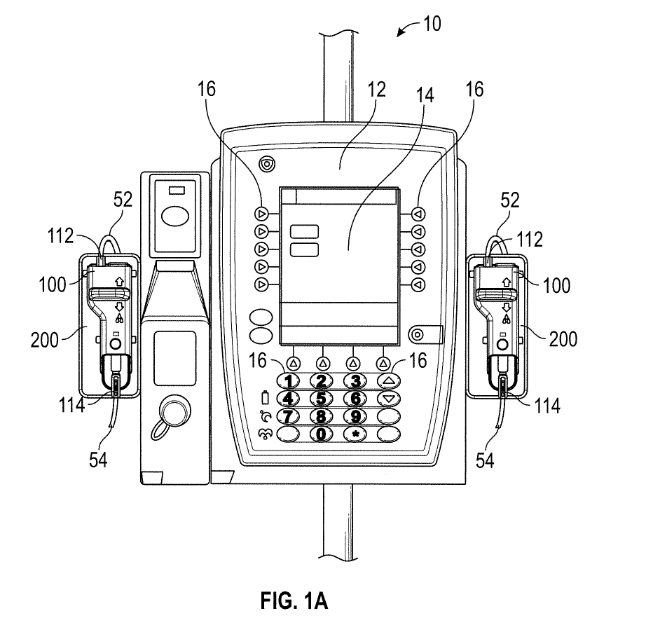

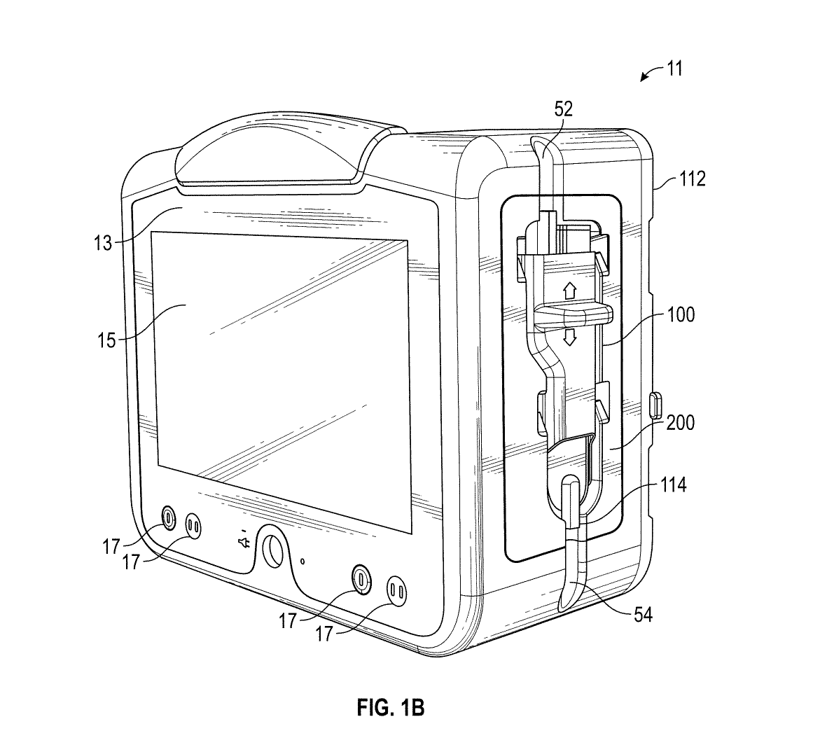

FIG. 1A illustrates an example of an infusion pump system that can contain an embodiment of a capacitive identification system. It is to be understood that this is only an exemplary infusion pump system, and a capacitive identification system can be utilized in any type of infusion pump system and/or in various other systems as discussed herein. The infusion pump system will be generally explained in reference to FIGS. 1A-3. An exemplary infusion pump system 10 may include central processing unit 12 with display screen 14 (e.g., touchscreen display), and data input features 16, for example, a keypad and a series of configurable buttons 16 adjacent to display screen 14. Other types of input and output devices may be used with central processing unit 12 and infusion pump system 10. In certain aspects, central processing unit 12 is operatively coupled to one or more interface modules, with cassette recesses 200, to control and communicate with various operational interfaces thereof.

FIG. 1B illustrates another example of an exemplary infusion pump system. This exemplary infusion pump system 11 may include one or more cassette recesses 200 and disposable IV pump cassettes 100. For example, cassette recess 200 may be configured to receive cassette 100 and provide various mechanical couplings and operational interfaces (e.g., fittings, motor, gearing, driveshaft, sensors, etc.). Infusion pump system 11 may include central processing unit 13 with display screen 15 (e.g., touchscreen display), and data input features 17, for example, a series of configurable buttons adjacent to display screen 15. In some implementations, the display screen 15 may provide a keypad or similar data entry feature. Other types of input and output devices may be used with central processing unit 13 and infusion pump system 11. In certain aspects, central processing unit 13 is operatively coupled to one or more interface modules, with cassette recesses 200, to control and communicate with various operational interfaces thereof.

In operation, an IV bag, syringe or other fluid source 52 may be fluidly connected to inlet 112 of cassette 100, and outlet 114 of cassette 100 may be fluidly connected to a patient 54 as shown in the examples of FIGS. 1A and 1B. Cassettes 100 may comprise a DEHP and Latex-free fluid pathway suitable for various patient populations (e.g., neonate, pediatric, and adult).

In operation, a user (e.g., a caregiver) may obtain a new disposable IV cassette 100 and prime cassette 100 before inserting cassette 100 into cassette recess 200. The caregiver may check for any visible air bubbles in the fluid pathway and may press on any accessible fluid reservoirs (e.g., pressure dome chambers) to move fluid through the cassette 100. Cassette 100 can be securely held and inserted into cassette recess 200 by, for example, a single hand of a caregiver. In this regard, a caregiver's other hand can be freed to perform other tasks.

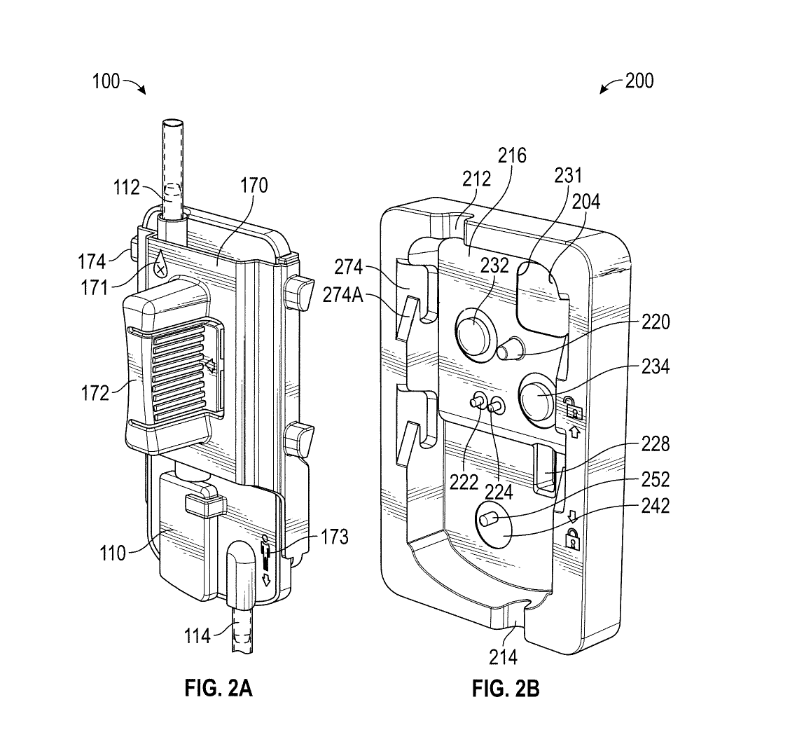

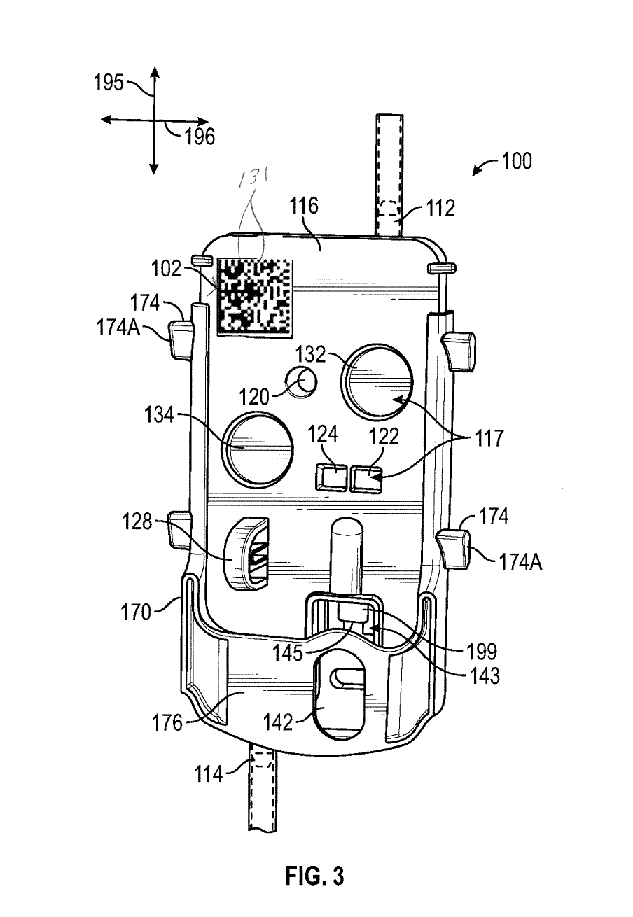

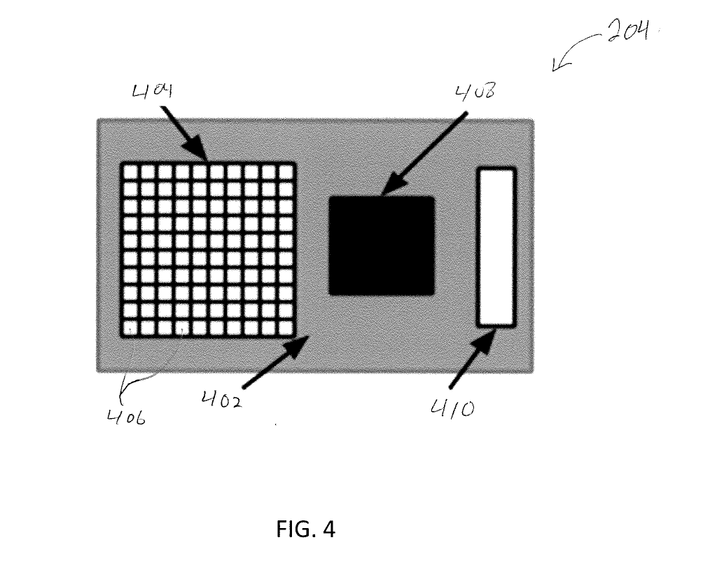

FIGS. 2A and 2B illustrate examples of a disposable IV pump cassette 100 and corresponding cassette recess 200 of an interface module. In accordance with certain embodiments, cassette 100 may comprise a cassette body 110 and a slider 170. Cassette 100 may include certain may include certain visual indicators related to operation aspects of the cassette and the infusion pump system in general. For example, cassette may include identifiable images such as fluid drops 171 indicating a position of slider 170 for free-flow (e.g., with a flow stop valve in an open position) and a patient FIG. 173 proximal to outlet 114. In accordance with some aspects, one or more cassette-seated sensors may be disposed within the cassette recess 200 so as to inform central processing unit 12 that the cassette is locked or secured into place within the cassette recess 200 or seat. For example, cassette recess may include a capacitive detector 204 such that a capacitive cassette identifier 102 (see, e.g., FIG. 3) can be capacitively detected to identify the cassette that has been mounted in cassette recess 200.

Cassette identifier 102 (e.g., a capacitive barcode formed from dielectric ink) may include one or more features 131 that include coded information such as, but not limited to, a manufacturer, type, serial number, expiration date, and use parameters of cassette 100 and/or a drug associated with cassette 100. Moreover, cassette identifier 102 may be disposed on a top half of the exterior surface of interface-facing frame portion 116 with respect to gravity during use. Thus, a bottom half of the exterior surface of interface-facing frame portion 116 can be reserved for a pump drive assembly and flow stop valve features, in accordance with certain embodiments.

As shown in FIG. 2B, capacitive detector 204 may be visually indicated by a border 231 formed on the surface of cassette recess 200 and may otherwise have a substantially planar outer surface that is easily wiped clean of liquid, dust, or other contaminants. Border 231 may be a printed border or a recess or ridge on the outer surface of an outer enclosure wall 216 of cassette recess 200. However, this is merely illustrative. In some embodiments, the outer surface of enclosure wall 216 may be substantially smooth and/or planar over detector 204 without an indicator of the location of the detector. In this way, ingress of liquid, dust, or other contaminants can be prevented by providing a cassette detector without any openings or structure interfaces (e.g., a detector formed behind a monolithic housing member that is free of interfaces between an optical (transparent) window and a housing structure as may be required for an optical barcode scanner).

Capacitive detector 204 may be configured to detect the size, shape, composition, density, or other aspects of features 131 of cassette identifier 102 when cassette 100 is installed in cassette recess 200. Dedicated processing circuitry (not explicitly shown in FIG. 2B) for capacitive detector 204 may be provided to readout and/or process capacitance signals from sensor elements in a capacitive sensor array for identification of a pattern (e.g., a one-dimensional barcode or a two dimensional barcode matrix) of cassette identifier 102. For example, the presence of a feature of a particular size, shape, or composition may change a rate of charge decay in various sensor elements of the sensor array in a predictable and measureable way. The dedicated processing circuitry and/or central processing unit 12 may be used to determine the size, shape, composition, density, or other aspects of features 131 based on the decay rates and to identify, for example, the barcode pattern and thus the cassette and an associated IV set (based on the barcode pattern).

IV disposable infusion sets may be categorized by therapy. For example, a syringe IV set may be used for neonatal intensive-care unit (NICU) procedures, an epidural IV set may be used to provide anesthesia, an oncology IV set may be used to provide chemotherapy (e.g., in a cancer ward or other cancer treatment setting), a large volume infusion IV set may be used to provide IV drugs in an emergency room (ER), operating room (OR), intensive care unit (ICU) or other procedure. Each IV set has different features that are relevant to the associated therapy and may be identified by capacitive detector 204 based on the pattern of features 131 of capacitive identifier 102.

Capacitive identifier 102 may be formed from, for example, a dielectric ink (e.g., an ink-jet or laser-jet ink formed from or infused with a dielectric material that can hold a charge and/or modify an electric field generated by detector 204). The dielectric ink may be printed directly onto the surface of cassette 100 to form identifier 102 or the dielectric ink may be printed on a label (e.g., an adhesive label) that is attached to cassette 100 after printing (as examples). The dielectric ink may have an appearance similar to conventional inks so that capacitive identifier 102 can also be detected using an optical scanner to provide interoperability between systems. In systems with particularly sensitive detectors, capacitive effects of patterned laser ablation films and/or conventional inks may also be detected and identified.

The dielectric ink may be printed to form a pattern of features 131 that is unique to each cassette, unique to an IV set associated with the cassette, or unique to a drug associated with the cassette (as examples). The pattern for each cassette may be stored along with identifying information of the cassette at, for example, a server that is communicatively connected to a capacitive detector so that, when the capacitive barcode is read, the detector or an associated device such as an infusion pump system can obtain identifying information for the cassette. Additionally, or alternatively, a capacitive barcode that is read using a capacitive detector may be stored by the device (e.g., by the infusion system) for recording of the use of that cassette and/or an associated drug or IV set.

Slider 170 can be fixably and slidably engaged with cassette body 110 such that slider 170 may articulate longitudinally with respect to cassette body 110, but will be constrained within range of sliding motion such that the slider remains coupled to the cassette body 110. Slider 170 may be formed from rigid plastic or polymer material and is clear or translucent in accordance with certain embodiments. In some embodiments, slider 170 may be polycarbonate. In accordance with certain aspects, slider 170 may be lockable at one or more positions, and may include a slider grip 172 for unlocking and articulating slider 170. Slider 170 may also include a plurality of protrusions 174 or lugs that are configured to mate and be releasably lockable with a plurality of slots 274 of the cassette recess 200 (e.g., L-shaped locking channels).

Each of the plurality of protrusions 174 may also comprise a flat face portion 174a that is configured to interface with a respective flat face ramp portion 274a of the cassette engagement slots 274. In this regard, cassette 100 can be self-guided and self-latched into the cassette recess 200. Accordingly, a door or lever action is not required in order to retain the cassette 100 within the cassette recess 200.

Additionally, an overall size of cassette 100 and cassette recess 200 may be reduced, in accordance with some aspects. For example, in certain embodiments, cassette body 110 may extended longitudinally a length between 70 mm and 90 mm. For orientation reference with respect to the various views of the examples illustrated of FIGS. 2A and 2B, longitudinal axis or y-axis 195 and latitudinal axis or x-axis 196 are provided as a reference on FIG. 3.

Various types, placement, and orientations of the plurality of protrusions 174 disposed on slider 170 are contemplated in the present disclosure. Aspects of the various cassette-coupling techniques illustrated in the example cassette embodiments described herein may be further combined and arranged into additional configurations suitable for specific implementations given the benefit of the present disclosure.

Cassette body 110 may comprise interface-facing frame portion 116 and slider-facing base portion (not shown) with membrane 117 disposed substantially therebetween. Portions of membrane 117 may extend through or be accessible from some openings of frame portion 116 (e.g., upstream pressure dome 132, downstream pressure dome 134, inlet-side valve 122, and outlet-side valve 124). In accordance with certain embodiments, membrane 117 can be a compliant material co-molded to the frame portion 116 and sealingly engaged with the base portion for defining a fluid pathway through cassette body 110 from inlet 112 to outlet 114. Mating edges of frame portion 116 and the base portion may be connected by fusing, welding, gluing, or the like. Membrane 917 and the base portion may further define a plurality of other features, some of which may be accessed through openings in frame portion 116.

Frame portion 116, membrane 117, and/or the base portion may define features in or along the fluid pathway, in accordance with certain embodiments. For example, beginning from inlet 112, the fluid pathway may include features such as, but not limited to, upstream pressure dome 132 (e.g., an inlet-side compliant reservoir), inlet-side valve 122, outlet-side valve 124, a pump chamber formed between valves 122 and 124, downstream pressure dome 134 (e.g., an outlet-side compliant reservoir), fluid pathway extension member 128, and a flow stop valve. Other features that are not in or along the fluid pathway, but are disposed on cassette body 110, may include positioning port 120 configured to receive cassette alignment protrusion 220.

In accordance with certain embodiments, membrane 117 may be formed from a thermoplastic elastomer (TPE). Characteristics of certain TPEs can enable effective co-molding with other materials, for example, polycarbonate. Accordingly, in some embodiments, membrane 117 may be co-molded to frame portion 116 and a striker may be co-molded to a portion of membrane 117 defining a flow stop valve 164. However, in some embodiments, membrane 117 can be formed from silicon, a silicon-based compound, an elastomeric material suitably compliant for fluid flow, or the like.

In accordance with certain embodiments, interface-facing frame portion 116 and a slider-facing base portion may be formed from a rigid plastic such as, but not limited, a polycarbonate. Additionally, the rigid plastic of frame portion 116 and the base portion may be clear or translucent. The material of membrane 117 (e.g., TPE or other compliant material) and rigid plastic slider 170 may also be clear or translucent, thereby allowing a user or caregiver to readily observe fluid passage through a substantial portion of the fluid pathway of cassette body 110. In some embodiments, the fluid pathway portion of cassette body 110 will be clear or translucent, and other portions will be frosted so as to direct a user or caregiver's attention to the fluid pathway.

In some implementations, slider 170, the base portion, and membrane 117 may be clear or translucent (or at least some portions along the fluid pathway), and the frame portion 116 may not be translucent. For example, the frame portion 116 may be colored in a manner so as to contrast against a color or tint of the fluid expected to be used with cassette 100. In some embodiments, a lens area may be disposed on the base portion alternatively, or in addition to, a lens area disposed on slider 170 to facilitate viewing of the fluid.

Pump drive interface 142 and pump actuator 242 may be configured as a reciprocating motion mechanism (e.g., a scotch-yoke configuration, a cam-driven (perpendicular motion) configuration, a linear actuator, a rotary actuator, etc.) in certain implementations. In such implementations, pump drive interface 142 may include opposing ramp portions for guiding a rotatable pin 252 of pump actuator 242 toward a slot of pump drive interface 142. The opposing ramp portions may allow self-alignment of the piston 145 to the pump interface pin 252. For example, the outer edges of the opposing ramp portions may be arranged at a distance that will ensure engagement with the rotatable pin 252 of pump actuator 242. When the rotatable pin 252 contacts one of the ramp portions, the pump drive interface 142 will move the piston to align the elongate slot of pump drive interface 142 with the rotatable pin 252 of pump actuator 242. However, it is to be appreciated that other pump drive assemblies are contemplated with cassette 100 and cassette recess 200 in accordance with the present disclosure. Actuator-receiving portion 142 may be accessible by pump actuator 242 via an aperture through interface-facing sider section 176

Piston 145 may be driven by a force provided by pin 252 against the sidewall surfaces of the elongate slot as pump actuator 242 rotates. The elongated configuration of the slot may allow pin 252 to reciprocate back and forth along the elongated dimension of the slot without providing a force on piston 145 in that direction as the pin provides a perpendicular force for actuating piston 145 within piston barrel 199. However, other configurations of a slot in interface 142 may be provided to generate various pumping characteristics with a rotating pin 252.

In some embodiments, pump drive assembly may be configured to produce a 3.5 mm piston stroke for operation with a pump chamber configured to be a 10 mm outer diameter reservoir. Moreover, the pump drive assembly may be arranged below the pump chamber, in accordance with some embodiments.

In certain embodiments, cassette recess 200 may include an upstream pressure-sensing probe 232 and downstream pressure sensing probe 234 enabling measurement of in-line pressure and fault isolation to a section of the fluid pathway. For example, upstream pressure sensing probe 232 may operably contact upstream pressure dome 132 through a corresponding opening of interface-facing frame portion 116. Similarly, downstream pressure sensing probe 234 may operably contact downstream pressure dome 134 through a corresponding opening of frame portion 116.

One or more fluid sensors may be disposed within sensor slot 228. The one or more fluid sensors disposed within sensor slot 228 can be ultrasonic sensors configured as an air-in-line detector, for example. In certain embodiments, extension member 128 may be disposed on cassette body 110 and positioned along the fluid pathway between downstream pressure dome 134 and a flow stop valve. However, in some embodiments, extension member 128 can be positioned at other locations along the fluid pathway such as, but not limited to, between inlet 112 and upstream pressure dome 132. Additionally, in other embodiments, a plurality of extension members 128 with a plurality of corresponding sensor slots 228 may be positioned along a fluid pathway of cassette body 110.

Cassette body 110, or a substantial portion thereof, may extend a depth of between 6 mm and 8 mm. Fluid pathway extension member 128 (see FIG. 3) may further extend between 8 mm to 10 mm. In certain aspects, the slider grip 172 of slider 170 may extend between 10 mm to 14 mm from cassette body 110. It is to be appreciated that the process of cleaning of inlet recess 212, outlet recess 214, and cassette recess 200 is made efficient in the shallow recess configuration in accordance with certain embodiments should any fluid or debris accumulate within cassette recess 200. The shallow recess configuration of cassette recess 200, and associated longitudinal alignment of cassette 100 such that a smaller of volumetric dimensions of cassette 100 (e.g., depth being smaller than length and width in certain embodiments) further enables additional space for arrangement of mechanical couplings and operational interfaces and optimizes the overall space requirements of cassette recess 200 and infusion pump system in general.

For example, a pumping operation of infusion pump system 10, 11 when cassette 100 is primed and seated in cassette recess 200 may comprise activating outlet-side valve actuator 224 such that outlet-side valve 124 is closed or sealed while activating inlet-side valve actuator 222 such that inlet-side valve 122 is opened. Opening of inlet-side valve 122 may coincide with or occur shortly before the start of a reverse stroke of piston 145 (e.g., a movement of piston 145 away from pump chamber). Accordingly, fluid can flow from upstream pressure dome 132 to the pump chamber. Alternatively, or in addition to, outlet-side valve 124 may comprise a one-way valve mechanism that permits flow of fluid under normal conditions in one direction (from a fluid container to a patient). Additionally, in some alternative embodiments, inlet-side valve 122 may also comprise a one-way valve or choke mechanism permitting flow of fluid in primarily one direction (e.g., from a fluid container to a patient) under normal operating conditions. In this configuration, cassette recess 200 may not need to incorporate either outlet-side valve actuator 224 or inlet-side valve actuator 222. Outlet-side valve 124 and inlet-side valve 122 may limit flow of fluid in one direction, but permit flow in an opposite direction in the event fluid pressure overcomes a cracking pressure of the valves.

Continuing with the valve-operated implementation, pumping operation may comprise activating outlet-side valve actuator 224 such that outlet-side valve 124 is open while activating inlet-side valve actuator 222 such that inlet-side valve 122 is closed or sealed. Opening of outlet-side valve 124 may coincide with or occur shortly before a start of a forward stroke of piston 145 (e.g., a movement of piston 145 toward the opening/access 125 of the pump chamber such that the volume of the pump chamber is reduced). Thus, fluid can flow from pump chamber down the fluid pathway to outlet 114.

In certain embodiments, the upstream pressure dome 132 may be smaller than the downstream pressure dome 134 to minimize retained volume. Likewise, the downstream pressure dome 134 may be larger than the upstream pressure dome 132 to improve resolution of fluid pressure thereby allowing for an accurate and precise volume of fluid to be pumped and any upstream or downstream pressures to be accurately measured.

Pump drive interface 142 can be operatively coupled to piston 145 slidably engaged within piston guide 143 and/or casing 199 (e.g., a generally cylindrical and/or frustoconical piston barrel) such that reciprocal movement of piston 145 within a pump chamber formed in part by the piston barrel 199 provides a moving seal that defines the edge of the pump chamber to urge fluid through the fluid pathway of cassette body 110.

When cassette 100 is installed in cassette recess 200, identifier 102 may be disposed in proximity to detector 204 so that one or more features 131 may be detected by their capacitive effect on a capacitive sensing array of detector 204.

FIG. 4 shows an example implementation of a capacitive detector 204 as described herein. As shown in FIG. 4, capacitive detector 204 may include a board 402 (e.g., a single or multi-layer printed circuit board or flexible printed circuit) on which a capacitive sensing array 404 is formed. In the example of FIG. 4, capacitive sensing array 404 is formed from an array of capacitive sensing elements 406 that are coupled to control circuitry 408 on the board. Control circuitry 408 may be a capacitive touch controller integrated circuit (IC) configured to energize (e.g., push charge into) individual cells 406 in the array 404. An electromagnetic field (e.g., an electromagnetic field controllably modulated by control circuitry 408 using sensing elements 406) may be projected, for example, through an enclosure wall (e.g., a plastic or other non-conductive housing wall) of the system. When a capacitive identifier 102 is placed within the electromagnetic field, the field may be detectably altered by the capacitive identifier based on the specific pattern of the identifier.

Control circuitry 408 may be further configured to readout capacitance signals from the sensing elements 406. For example, control circuitry 408 may monitor a decay rate of the induced charge on neighboring cells and determine a capacitance change in each cell caused by the presence of a dielectric material (e.g., a portion of a capacitive barcode) in proximity to that cell. Control circuitry 408 may provide analog or digitized capacitance measurement signals, a decoded bit stream, and/or a barcode image to the central processing unit of infusion pump system 10 (e.g., via a connector 410 that communicatively couples board 402 to, for example, a main board that includes the central processing unit). The central processing unit, other dedicated cassette identification circuitry in the system, or control circuitry 408 may identify the pattern of the identifier 102 and, based on the identified pattern, identify the installed cassette.

Identifying the installed cassette may include obtaining a unique cassette identifier (e.g., a serial number), a cassette type, an IV set type associated with the cassette, an expiration date or other cassette information. The cassette information may be obtained by locally decoding coded information in the identified pattern and/or accessing a database of information stored in connection with identifier patterns or with serial numbers of particular cassettes.

For example, the serial number of a cassette and an expiration date of the cassette may be decoded by processing circuitry of the pump system. Alternatively or additionally, a database (e.g., a remote network database) of information associated with a decoded serial number may be accessed to obtain cassette information (e.g., recall information or operating parameters for a particular cassette or cassette type).

Although a rectangular circuit board 402 is shown in FIG. 4, board 402 may have one or more features such as openings and cutouts that help align and accommodate installation of board 402 in another device such as behind a housing enclosure of system 10 in cassette recess 200. The housing enclosure may be opaque. In various implementations, sensing elements 406 may be formed by individual charge storage elements (e.g., conductive pads coupled to traces in board 402) or may be formed by the intersection of conductive traces such as perpendicularly oriented sensing lines and drive lines on the board. Capacitive sensing array 404 may be etched in copper on board 402.

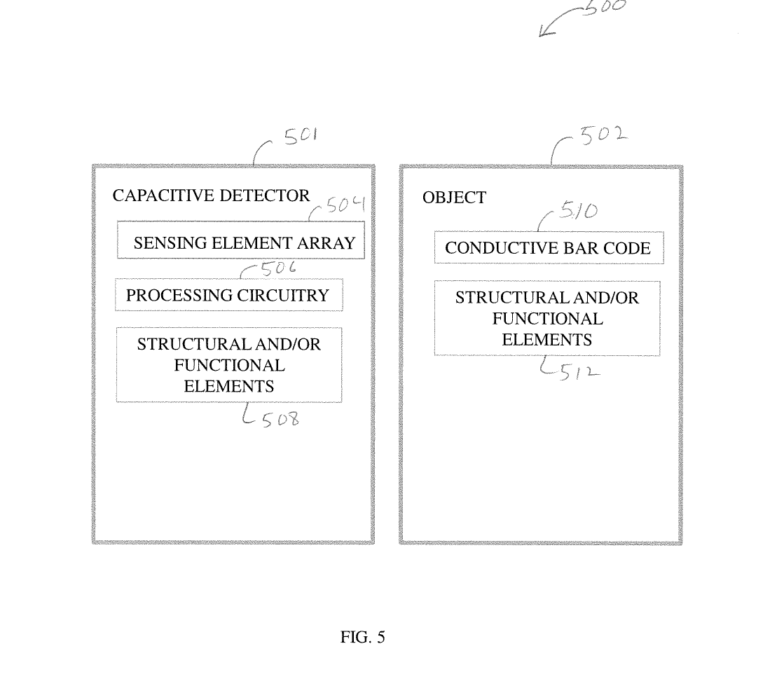

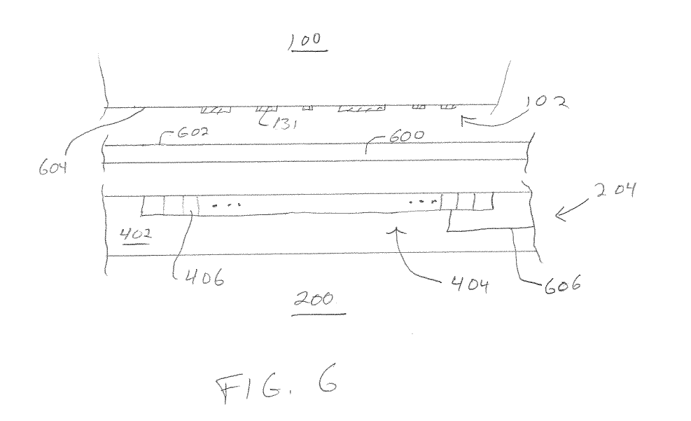

Although identification of IV sets by capacitive identification of a cassette placed in a cassette recess of an infusion pump system is sometimes discussed herein as an example, it should be appreciated that capacitive object identification using a capacitive barcode can be implemented for various other types of systems. FIG. 5 is a block diagram illustrating a system 500 having a capacitive detector and an object having a capacitive identifier.

As shown in FIG. 5, system 500 may include capacitive detector 501 and one or more objects such as object 502 having a capacitive identifier such as capacitive barcode 510. Capacitive barcode 510 may, for example, be implemented as identifier 102 of a cassette for an infusion pump system or may be implemented as an identifier for another object. For example, object 502 may be a consumer product such as a grocery item, a clothing item, a hardware item, an electronic device, a household item, an automotive item, or the like. Capacitive barcode 510 may be attached to or integrally formed with, for example, packaging of the object or the object itself. For example, capacitive barcode 510 may be a printed pattern of dielectric ink on or within a shipping container for tracking of the production, sales, shipping, and/or usage of the contents of the container and, if desired, a specific produce, a type of product, or a brand of product in the container (as examples). As used herein, a shipping container may indicate a container for transport on a cargo ship, an airplane, a train car, or a truck or may indicate cardboard, plastic, or other packaging for an individual product.

In the example in which object 502 is a consumer product, capacitive detector 501 may be implemented as a product scanner at a warehouse, a manufacturing facility, a retail location, or any other location at which it may be desirable to identify the location, type, brand, or other aspect of the consumer product. In one example, capacitive detector 501 may be provided at the checkout stand at a grocery store and configured to use sensing element array 504 to detect one or more features of a capacitive barcode 510 that is or is not optically visible on the object from the detector.

One or more features of capacitive barcode 510 may be considered to be in the proximity of a sensing element array 504 when the one or more features are within a perpendicular distance of less than 1 cm, less than 3 cm, less than 5 cm, less than 10 cm, between 1 mm and 5 cm, greater than 0.5 mm, greater than 10 mm, or between 10 mm and 5 cm (as examples).

As shown in FIG. 5, capacitive detector 501 may include processing circuitry 506 (e.g., one or more processors, integrated circuits, volatile or non-volatile memory, etc. such as control circuitry 408 of FIG. 4) for determining capacitance values and for extracting identifying information for an object based on the determined capacitance values. For example, processing circuitry 506 may store a decoding key with which an object serial number can be decoded from a detected pattern in the capacitive barcode. Processing circuitry 506 may store, or maintain remote access to, a look-up table of object serial numbers and additional object information (e.g., product, cassette, medicine, expiration, brand, manufacturer, location or other information) associated with each serial number. As shown, object 502 may include structural and/or functional elements 512 such as a housing, packaging, electronic devices, or other structural and/or functional elements according to various embodiments. For example, in one embodiment, structural and/or functional elements 512 may include a food package and the food inside the food package. In another embodiment, structural and/or functional elements 512 may include a housing of a cassette such as cassette 100 and the fluid pathways, valves, and pump components therein.

Capacitive detector 501 may include structural and/or functional elements 508 such as a housing, packaging, electronic devices, or other structure and/or functional elements according to various embodiments. For example, in one embodiment, structural and/or functional elements 508 may include a conveyor belt of a grocery checkout stand, a detector housing, and additional processing circuitry such as checkout, payment, and/or inventory circuitry. Elements 508 may include actuating components for scanning the sensor array over an area or a volume of interest to identify and capture the barcode pattern when the precise location of the identifier is not constant. In another embodiment, structural and/or functional elements 508 may include a housing of a infusion pump system 10, a cassette recess, and alignment, valve operation, and pump drive components therein. Although the capacitive identifier of object 502 is described herein as a printed barcode, it should be appreciated that a patterned dielectric film or other patterned dielectric identifier for object 502 may be provided that can be scanned or read based on capacitance values obtained by detector 501. Capacitive detector 501 may be configured to read stationary and/or moving capacitive codes.

A capacitive detector for reading a capacitive identifier 102 may be formed within a housing of another device in some embodiments. FIG. 6 shows a capacitive detector 204 disposed within an opaque outer housing structure 600 of a device so that the outer housing structure 600 forms an outer surface 602 of the device. In the example of FIG. 6, board 402 containing capacitive sensing array 404 is disposed within the opaque housing of the device. For example, housing structure 600 may be an outer housing structure of cassette recess 200 of infusion pump system 10 and board 402 may be disposed behind the housing structure of the cassette recess. Board 402 and sensing array 404 may be positioned, and the mounting features for mounting the cassette in the cassette recess (e.g., features 174 of cassette 100, features 274 of cassette recess 200, and guide pin 220) may be configured such that, when the cassette is installed in the cassette recess, the capacitive identifier of the cassette is located at a distance from sensing elements 406 at which the capacitive features 131 generate a detectable change in the readout of sensing elements 406 (e.g., by altering an electromagnetic field that is generated by sensing elements 406 and projects through housing member 600). Various conductive traces such as conductive trace 606 may be provided on and/or within board 402 for communicatively coupling sensing elements 406 to control circuitry and/or external connectors coupled to the board.

In the example of FIG. 6, features 131 of capacitive identifier 102 are shown as dielectric ink printed on and projecting from the outer surface 604 of cassette 100. However, this is merely illustrative. In other embodiments, the capacitive pattern of identifier 102 can be formed on or embedded within an adhesive label that is attached to outer surface 604, can be printed on an internal surface of cassette 100 or can be embedded within a structure such as housing member 600 of cassette 100. Forming the capacitive pattern on an internal surface or embedded within a label or a housing structure can be advantageous in situations in which it is preferable to have an identifier that is not visible from the outside of the product to increase the difficultly of copying or counterfeiting the identifier.

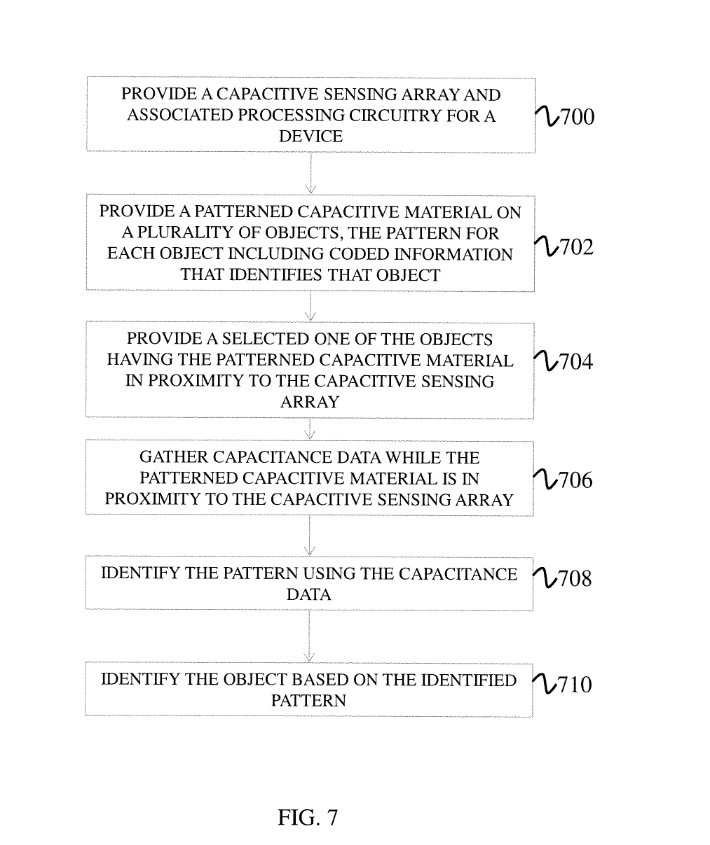

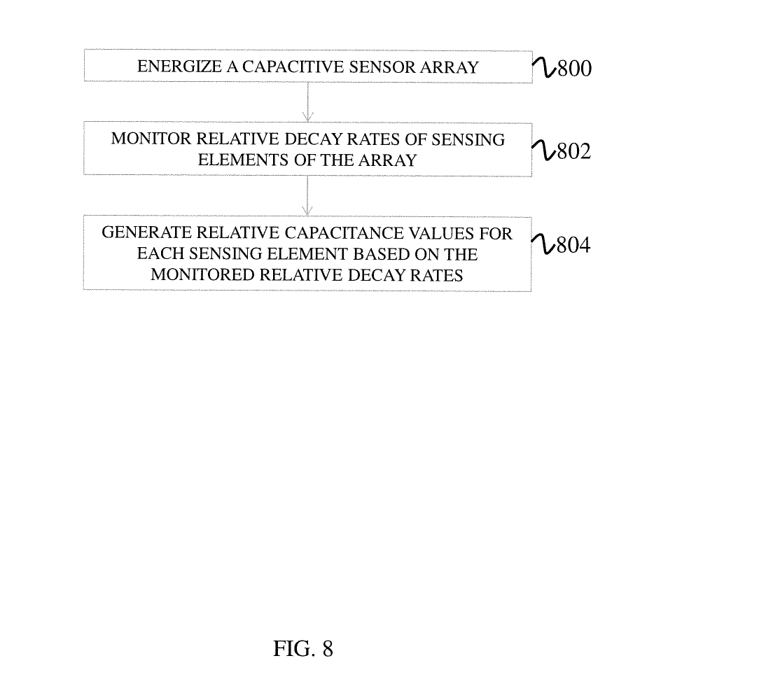

Illustrative operations that may be performed for capacitive identification of an object are shown in FIG. 7, according to an embodiment.

At block 700, a capacitive sensing array and associated processing circuitry may be provided for a device. As examples, the device may be an infusion pump system or a product scanning system for a retailer, warehouse, shipper, or manufacturer. The capacitive sensing array may include a plurality of sensing elements formed on a common circuit board with the processing circuitry. The circuit board may be provided within an outer housing enclosure of the device to prevent ingress of moisture, dust or other contaminants into the device without the need for special sealing for, for example, an optical window for an optical barcode scanner camera.

At block 702, a patterned capacitive material (e.g., a printed pattern of dielectric ink) may be provided on each of a plurality of objects. The pattern for each object may include coded information such as a serial number and/or expiration date coded into the pattern and specific to that object. The plurality of objects may include any objects for which identification may be desired, various examples of which have been discussed herein (e.g., a pump cassette for an infusion pump system).

At block 704, a selected one of the objects have the patterned capacitive material may be provided in proximity to the capacitive sensing array. In one example, providing the selected one of the objects in proximity to the capacitive sensing array may include installing a pump cassette in a corresponding cassette recess of an infusion pump system such that the patterned capacitive material is disposed within an electromagnetic field generated by the capacitive sensing array. In another example, providing the selected one of the objects in proximity to the capacitive sensing array may include (a) placing a product to be purchased on a scanner having the capacitive sensing array where the product has the patterned capacitive material disposed on packaging of the product and (b) scanning the capacitive sensing array to move an electromagnetic field generated by the capacitive sensing array into the proximity of the patterned capacitive material.

At block 706, capacitance data may be gathered by the capacitive sensing array while the patterned capacitive material is in proximity to the capacitive detector. Gathering capacitance data may include recording a change in the capacitance of each of a plurality of capacitive sensing elements that is caused by the presence of the patterned capacitive material.

At block 708, the pattern on the selected object may be identified using the capacitance data. The pattern may be a barcode, a quick response (QR) code or other coded pattern that can be decoded to extract data from the pattern and/or can be used to access locally or remotely stored information associated with that pattern. Identifying the pattern may include generating and/or storing an image of the pattern based on the capacitance data.

At block 710, the object may be identified based on the identified pattern. For example, a serial number of the object may be extracted from the identified pattern by decoding the pattern. In another example, the identified pattern may be compared to a database of stored patterns to obtain object identifying information that is stored in the database in connection with the stored pattern.

Illustrative operations that may be performed for gathering capacitance data as described above in connection with block 706 of FIG. 7 are shown in FIG. 8, according to an embodiment.

At block 800, a capacitive sensing array that is coupled to processing circuitry for a device may be energized. For example, energizing the array may include pushing a known amount of charge onto each of a plurality of sensing elements in the array. The energized array may generate an electromagnetic field that extends out of the device through a housing enclosure of the device. The patterned capacitive material may be disposed in the electromagnetic field and capacitive effects of the patterned capacitive material may alter the capacitance of various capacitive sensing elements in the array.

At block 802, the decay rates of the charges on the sensing elements in the array may be monitored (e.g., by sampling the amount of charge on each element over a period of time). Due to the altered capacitances caused by the patterned capacitive identifier, the decay rates of various sensing elements may be different from (a) the known decay rate, in isolation from a capacitive identifier, of that element and/or (b) the measured concurrent decay rates of other sensing elements in the array.

At block 804, relative capacitance values (and/or calibrated absolute capacitance values) may be generated for each sensing element based on the monitored relative decay rates. The capacitance values may be stored, processed (e.g., filtered, amplified, and/or digitized), and/or output by the processing circuitry for identification of the pattern.

The subject technology is illustrated, for example, according to various aspects described above. Various examples of these aspects are described as numbered concepts or clauses (1, 2, 3, etc.) for convenience. These concepts or clauses are provided as examples and do not limit the subject technology. It is noted that any of the dependent concepts may be combined in any combination with each other or one or more other independent concepts, to form an independent concept. The following is a non-limiting summary of some concepts presented herein:

Concept 1. An apparatus, comprising:
a capacitive sensing array; and
processing circuitry coupled to the capacitive sensing array and configured to:
   operate the capacitive sensing array to generate an electromagnetic field; and
   determine an identity of an object based on capacitance values generated by the capacitive sensing array when a capacitive identifier of the object is placed within the electromagnetic field generated by the array.

Concept 2. The apparatus of Concept 1 or any other Concept, further comprising a housing enclosure, wherein the capacitive sensing array is disposed within the housing enclosure.

Concept 3. The apparatus of Concept 2 or any other Concept, wherein the housing enclosure is opaque.

Concept 4. The apparatus of Concept 1 or any other Concept, wherein the capacitive identifier comprises a patterned dielectric ink.

Concept 5. The apparatus of Concept 1 or any other Concept, wherein the capacitive sensing array and the processing circuitry are disposed on a common printed circuit.

Concept 6. The apparatus of Concept 5 or any other Concept, wherein capacitive sensing array comprises an array of sensing elements etched in copper on the printed circuit.

Concept 7. The apparatus of Concept 6 or any other Concept, wherein the processing circuitry is configured to identify the object based on the relative capacitance of each of the sensing elements.

Concept 8. The apparatus of Concept 1 or any other Concept, wherein the processing circuitry is configured to identify the object based on coded information in the capacitive identifier.

Concept 9. A system comprising the apparatus of Concept 1 or any other Concept and the capacitive identifier of the object.

Concept 10. The system of Concept 9 or any other Concept, wherein the apparatus comprises a cassette recess of an infusion pump system, wherein the object comprises a pump cassette coupled to an intravenous fluid set, and wherein the capacitive identifier is disposed on the cassette.

Concept 11. A pump cassette, comprising:

a rigid body comprising a compliant membrane that defines a controllable fluid pathway that extends from an inlet port to an outlet port; and

a capacitive identifier comprising a coded pattern that identifies the pump cassette.

Concept 12. The pump cassette of Concept 11 or any other Concept, wherein the capacitive identifier comprises a dielectric ink printed on an outer surface of the pump cassette.

Concept 13. The pump cassette of Concept 12 or any other Concept, wherein the dielectric ink forms a one-dimensional capacitive barcode.

Concept 14. The pump cassette of Concept 12 or any other Concept, wherein the dielectric ink forms a two-dimensional capacitive barcode matrix.

Concept 15. The pump cassette of Concept 11 or any other Concept, wherein the coded pattern comprises a coded serial number for the pump cassette.

Concept 16. An infusion pump system, comprising:

a processing unit; and

a cassette recess adapted to receive a pump cassette, the cassette recess comprising:
a plurality of mechanisms operably coupled to the processing unit and configured to control fluid flow in the pump cassette; and
a capacitive detector configured to capacitively detect and identify the pump cassette.

Concept 17. The infusion pump system of Concept 16 or any other Concept, further comprising an opaque housing enclosure, wherein the capacitive sensing array is configured to detect a capacitive identifier on the pump cassette through the opaque housing enclosure.

Concept 18. The infusion pump system of Concept 17 or any other Concept, wherein the processing unit is configured to identify the pump cassette based on the detected capacitive identifier.

Concept 19. The infusion pump system of Concept 18 or any other Concept, further comprising the pump cassette, wherein the capacitive detector is configured to identify an IV set type based on detected capacitive identifier.

Concept 20. The infusion pump system of Concept 16 or any other Concept, wherein the plurality of mechanisms comprises a plurality of actuators configured to operate a piston and plurality of valves of the pump cassette.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

One or more aspects or features of the subject matter described herein may be realized in digital electronic circuitry, integrated circuitry, specially designed ASICs (application specific integrated circuits), computer hardware, firmware, software, and/or combinations thereof. For example, infusion pump systems disclosed herein may include an electronic system with one or more processors embedded therein or coupled thereto. Such an electronic system may include various types of computer readable media and interfaces for various other types of computer readable media. Electronic system may include a bus, processing unit(s), a system memory, a read-only memory (ROM), a permanent storage device, an input device interface, an output device interface, and a network interface, for example.

Bus may collectively represent all system, peripheral, and chipset buses that communicatively connect the numerous internal devices of electronic system of an infusion pump system. For instance, bus may communicatively connect processing unit(s) with ROM, system memory, and permanent storage device. From these various memory units, processing unit(s) may retrieve instructions to execute and data to process in order to execute various processes. The processing unit(s) can be a single processor or a multi-core processor in different implementations.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. An apparatus, comprising:
a capacitive sensing array; and
processing circuitry coupled to the capacitive sensing array and configured to:
operate the capacitive sensing array to generate an electromagnetic field; and
determine an identity of an object based on capacitance values generated by the capacitive sensing array when a capacitive identifier of the object is placed within the electromagnetic field generated by the array.

2. The apparatus of claim 1, further comprising a housing enclosure, wherein the capacitive sensing array is disposed within the housing enclosure.

3. The apparatus of claim 2, wherein the housing enclosure is opaque.

4. The apparatus of claim 1, wherein the capacitive identifier comprises a patterned dielectric ink.

5. The apparatus of claim 1, wherein the capacitive sensing array and the processing circuitry are disposed on a common printed circuit.

6. The apparatus of claim 5, wherein capacitive sensing array comprises an array of sensing elements etched in copper on the printed circuit.

7. The apparatus of claim 6, wherein the processing circuitry is configured to identify the object based on the relative capacitance of each of the sensing elements.

8. The apparatus of claim 1, wherein the processing circuitry is configured to identify the object based on coded information in the capacitive identifier.

9. A system comprising the apparatus of claim 1 and the capacitive identifier of the object.

10. The system of claim 9, wherein the apparatus comprises a cassette recess of an infusion pump system, wherein the object comprises a pump cassette coupled to an intravenous fluid set, and wherein the capacitive identifier is disposed on the cassette.

11. A pump cassette, comprising:
a rigid body comprising a compliant membrane that defines a controllable fluid pathway that extends from an inlet port to an outlet port; and
a capacitive identifier comprising a coded pattern that identifies the pump cassette.

12. The pump cassette of claim 11, wherein the capacitive identifier comprises a dielectric ink printed on an outer surface of the pump cassette.

13. The pump cassette of claim 12, wherein the dielectric ink forms a one-dimensional capacitive barcode.

14. The pump cassette of claim 12, wherein the dielectric ink forms a two-dimensional capacitive barcode matrix.

15. The pump cassette of claim 11, wherein the coded pattern comprises a coded serial number for the pump cassette.

16. An infusion pump system, comprising:
a processing unit; and
a cassette recess adapted to receive a pump cassette, the cassette recess comprising:
a plurality of mechanisms operably coupled to the processing unit and configured to control fluid flow in the pump cassette; and
a capacitive detector configured to capacitively detect and identify the pump cassette.

17. The infusion pump system of claim 16, further comprising an opaque housing enclosure, wherein the capacitive sensing array is configured to detect a capacitive identifier on the pump cassette through the opaque housing enclosure.

18. The infusion pump system of claim 17, wherein the processing unit is configured to identify the pump cassette based on the detected capacitive identifier.

19. The infusion pump system of claim 18, further comprising the pump cassette, wherein the capacitive detector is configured to identify an IV set type based on detected capacitive identifier.

20. The infusion pump system of claim 16, wherein the plurality of mechanisms comprises a plurality of actuators configured to operate a piston and plurality of valves of the pump cassette.
